(19) United States
(12) Patent Application Publication
(10) Pub. No.: US 2022/0298483 A1
(43) Pub. Date: Sep. 22, 2022

(54) METHOD AND COMPOSITION FOR CHONDROGENESIS

(71) Applicant: National Health Research Institutes, Zhunan Township (TW)

(72) Inventors: Lin-Ju YEN, Zhunan Township (TW); Chen-Chan HSIEH, Zhunan Township (TW)

(73) Assignee: National Health Research Institutes, Zhunan Township (TW)

(21) Appl. No.: 17/697,168

(22) Filed: Mar. 17, 2022

Related U.S. Application Data
(60) Provisional application No. 63/162,778, filed on Mar. 18, 2021.

(51) Int. Cl. C12N 5/0775; C12N 5/077; A61P 19/02; A61K 35/32

(52) U.S. Cl. CPC C12N 5/0662; C12N 5/0655; A61P 19/02; A61K 35/32; C12N 2506/1346; C12N 2501/415

(57) ABSTRACT

The present application provides a composition for inducing chondrogenesis comprising an Wnt antagonist and a pharmaceutically acceptable carrier. The present application also provides a method for inducing chondrogenesis comprising administering an Wnt antagonist and a pharmaceutically acceptable carrier to a subject. The present application further provides a method for treating a cartilage-related disease comprising administering a therapeutically effective amount of an Wnt antagonist and a pharmaceutically acceptable carrier to a subject.

### BACKGROUND OF THE INVENTION

### 1. Field of the Invention

The present invention relates to a composition and a method for inducing chondrogenesis.

### 2. Description of the Related Art

Cartilage and joint diseases are one of the most common clinical conditions affecting quality of life worldwide, due to increases in the two major risk factors of age and obesity. Current treatments for arthritic diseases are largely limited to symptom palliation using analgesics for mild cases, and immunosuppressants and/or surgery for more serious cases. However, these treatments are not disease-modifying or curative; moreover, cartilage is unable to regenerate. While more novel treatment using autologous cartilage tissue or chondrocytes are being tested, the rarity of the cell/tissue source, ex vivo culture difficulties, and low cell viability after transplantation have resulted in limited success with these methods. Thus, continued investigation into more efficacious methods of regenerating cartilage is ongoing to find curative treatments for these common and debilitating diseases.

Human multipotent mesenchymal stem cells (MSCs) are versatile somatic stem cells with immunomodulatory properties. First isolated in adult bone marrow (BM), MSCs have subsequently been found in numerous post-natal organ/tissues as well as directly differentiated from pluripotent stem cells such as human embryonic stem cells (ESCs) (Barberi et al., 2005; Yen et al., 2009) and induced pluripotent stem cells (iPSCs) (Kimbrel et al., 2014; Wang et al., 2018). MSCs readily differentiate into chondrocytes, osteoblasts, adipocytes, and fibroblasts, and their easy accessibility compared to many other cell types including chondrocytes render them ideal for use in cartilage-related diseases. However, chondrogenic differentiation efficiency is low, due to the requisite cumbersome 3-dimensional (3D) pellet culture and the relative lack of knowledge on molecular mechanisms involved in chondrogenesis, compared to osteogenesis and adipogenesis. The most common factors used to induce MSC chondrogenesis are TGFβ1 and TGFβ3, two pleomorphic growth factors, but information on mechanisms involved for either of these factors are surprisingly scarce despite decades of use (Wang et al., 2014). Moreover, TGFβ is well known to induce fibrosis, as well as ossification in cartilage (Chen et al., 2012; Kurpinski et al., 2010). It is difficult to avoid osteogenic specification with use of proteins from this family during chondrogenic differentiation.

Thus, there clearly is a need for more precision and efficiency in achieving MSC chondrogenesis for therapeutic application.

### SUMMARY

The present application describes a composition for inducing chondrogenesis comprising an Wnt antagonist and a pharmaceutically acceptable carrier.

The present application also provides a method for inducing chondrogenesis comprising administering an Wnt antagonist and a pharmaceutically acceptable carrier to a subject.

The present application further provides a method for treating a cartilage-related disease comprising administering a therapeutically effective amount of an Wnt antagonist and a pharmaceutically acceptable carrier to a subject.

### BRIEF DESCRIPTION OF THE DRAWINGS

FIG. 1 shows that Wnt/β-catenin antagonism enhances human MSC chondrogenesis while agonism suppress chondrogenesis and upregulate master osteogenic transcription factor RUNX2. (A) Alcian Blue staining (left panel) of 3D pellet-cultured human induced pluripotent stem cell-derived MSCs (iPSC-MSCs), embryonic stem cell-derived MSCs (ESC-MSCs), and bone marrow-MSCs (BMMSCs) treated with 10 μM of either the Wnt/β-catenin antagonist XAV939 (XAV) or the agonist CHIR99021 (CHIR) in complete chondrogenic medium containing TGFβ3 (ChM) for 20 days. Quantification of Alcian Blue staining (right graph) was performed, with comparisons of Wnt antagonism or agonism to ChM for each MSC type. (B) Alcian Blue staining (left panel) and quantification (right panel) of pellet-cultured iPSC-MSCs treated with either XAV or CHIR at the indicated concentrations in ChM for 20 days. (C-F) Gene expression levels of the chondrogenic genes (C) SOX9, (D) collagen 2A1 (COL2A1), and (E) aggrecan (ACAN), as well as (F) RUNX2, the osteogenic master transcription factor, in 3D pelletcultured iPSC-MSCs treated with either XAV or CHIR in ChM cultured for 3 days as analyzed by quantitative PCR (qPCR). *, p<0.05, **, p<0.01, ***, p<0.001.

FIG. 2 shows that gene set of signaling by TGFβ family are enriched in human smooth muscle cells compared to MSCs, and TGFβ rapidly increases α-smooth muscle actin (αSMA) and RUNX2 expression in MSCs during chondrogenic induction. (A) GSEA enrichment plot of Signaling by TGFB family members in human BM-MSCs (MSC; GSE128949) compared to smooth muscle cells (SMC; GSE109859). The normalized enrichment score (NES) and nominal p values are shown. (B) Relative changes of mean expression levels of selected relevant genes and the p value in human primary SMCs compared to BM-MSCs (SMC vs. MSC). Genes with significant upregulation (p<0.05, fold-change>1) are colored in red. (C) Confocal immunofluorescence detection (scale bar, 20 μm) and (D) quantification of αSMA on Day 3 of pellet-cultured iPSC-MSCs in ChBM alone or with addition of TGFβ1 or TGFβ3; nuclei are labeled by 4′,6-diamidino-2-phenylindole (DAPI) and αSMA fluorescent intensity was normalized to nuclear staining (DAPI). (E) Gene expression levels of RUNX2 as quantified by qPCR on Day 3 of pellet-cultured iPSC-MSCs in ChBM alone or with addition of TGFβ1 or TGFβ3. *, p<0.05, **, p<0.01, ***, p<0.001.

FIG. 3 shows that Wnt/β-catenin antagonism alone induced more rapid MSC chondrogenesis than TGFβ in vitro and in vivo. (A) Alcian Blue staining of pellet-cultured iPSC-MSCs treated with the indicated modulators (10 ng/mL TGFβ3, 10 μM CHIR, or 10 μM XAV) at Day 20 and Day 10 (left top and bottom panels, respectively), with absorbance quantification for Day 10 results (right panel). (B) Alcian Blue staining (left panel) and absorbance quantification (right panel) of micromass-cultured iPSC-MSCs treated with the indicated modulators for 10 days. (C and D) Expression levels of chondrogenic gene (C) COL2A1 and (D) ACAN in 3-day pellet-cultured MSCs treated with the indicated modulators for 3 days as quantified by qPCR. (E) Schematic procedure of in vivo experimentation. Mouse BM-MSCs were cultured as 3D pellets in ChBM first then transplanted subcutaneously into wild type mouse. Modulators (10 μM of CHIR, XAV, or 10 ng/mL of TGFβ3) were injected locally every 3 days till harvest at Day 20. (F) Alcian Blue staining (left panel) and absorbance quantification (right panel) of harvested tissue sections from transplanted MSCs treated with the indicated modulators at Day 20. *, p<0.05, **, p<0.01, ***, p<0.001. *, p<0.05, **, p<0.01, ***, p<0.001.

FIG. 4 shows that Wnt/β-catenin antagonism but not TGFβ agonism during MSC chondrogenesis decreased canonical Wnt/β-catenin transcriptional activity including RUNX2 expression. (A) Immunofluorescent staining of β-catenin in iPSC-MSC pellets cultured in ChBM only or with addition of TGFβ3 (10 ng/mL), CHIR (10 μM), or XAV (10 μM) at 1 day. Dotted line indicates nuclear borders (stained with DAPI). Scale bar, 10 μm (B) Quantification of nuclear β-catenin intensity. (C-D) Gene expression levels of (C) AXIN2 and (D) RUNX2 in iPSC-MSC pellets cultured in ChBM only or with addition of TGFβ3 (10 ng/mL), CHIR (10 μM), or XAV (10 μM) for 3 days as quantified by qPCR. #, compared to all other groups. *, p<0.05, **, p<0.01, ***, p<0.001.

FIG. 5 shows that Wnt/β-catenin antagonism but not TGFβ agonism increases N-cadherin expression, and interactions with β-catenin as well as actin cytoskeleton-mediated condensation. (A) Representative confocal immunofluorescence microscopy images and (B) quantification of N-cadherin expression in iPSC-MSCs cultured as micromass in ChBM only or with addition of TGFβ3 (10 ng/mL), CHIR (10 μM), or XAV (10 μM) for 1 day. Nuclei are labeled by DAPI. Scale bar, 5 μm. (C) Proximity ligation assay (PLA) for N-cadherin-β-catenin (Ncad-βcat) interaction and (D) quantification of signal counts per field of iPSC-MSCs cultured as micromass in the indicated chondrogenic conditions for 1 day. Nuclei are labeled by DAPI. Yellow scale bar, 20 μm. White scale bar, 5 μm. (E) Relationship of core elements in N-cadherin pathway derived from the Pathway Interaction Database. PID_NCADHERIN_PATHWAY filtered by first neighborhood of CTNNB1 and CDH2 are presented. (F) Images of phase contrast and Alcian Blue staining images (top and bottom panel respectively) and (G) absorbance quantification of pellet-cultured iPSC-MSCs treated with the indicated modulators (0.25 μM cytochalasin D (CytoD), 10 μM XAV) at Day 10. *, p<0.05, **, p<0.01, ***, p<0.001.

FIG. 6 shows that Wnt/β-catenin and TGFβ-related pathways are significantly downregulated in human primary chondrocytes compared to MSCs and osteoblasts. (A) Principal component analysis (PCA) based on transcriptomic data and (B) interpreted relationship between human primary chondrocytes (Chondro), osteoblasts (Ostb) and BM-MSCs (MSC). (C) Enrichment analysis of major developmental pathways in human primary chondrocytes compared to MSCs (Chondro v.s. MSC), osteoblasts compared to MSCs (Ostb v.s. MSC) and chondrocytes compared to osteoblasts (Chondro v.s. Ostb) by Gene Set Enrichment Analysis (GSEA). Pathways in blue and orange are significantly enriched (p<0.05) with negative and positive normalized enrichment score respectively, non-significant pathways (p>0.05) were colored in grey. (D) GSEA enrichment plot of signaling by Wnt (stable Identifier: R-HSA-195721) and TGF family members (stable Identifier: R-HSA-9006936) in human primary chondrocytes compared to MSCs (left 2 panels) and osteoblasts (right 2 panels). The normalized enrichment score (NES) and nominal p values are shown. (E) Heatmap showing the Robust Multi-array Average (RMA)-normalized expression levels of selected genes relevant to osteogenesis (Osteo), hypertrophic cartilage (Hypertrophy), chondrogenesis (Chondro), inhibition of Wnt/β-catenin signaling (Wnt/β-catenin inhibition) and activation of Wnt/β-catenin signaling (Wnt/β-catenin activation) in samples of human primary chondrocytes, BMMSCs and osteoblasts. (F) Relative changes of mean expression levels of selected gene sets and the p value in human primary chondrocytes compared to MSCs and chondrocytes compared to osteoblasts. Genes with significant upregulation (p<0.05, fold-change>1) are colored in red, and with significant downregulation (p<0.05, fold-change<−1) are colored in green. (G) Upstream analysis for differential gene expression of human primary chondrocytes compared to MSCs (Chondro vs. MSC, top panel) and compared to osteoblasts (Chondro vs. Ostb, bottom panel) generated by Ingenuity Pathway Analysis (IPA). Wnt-, TGFβ- and adherens junction-related factors with significance (p<0.05) are presented.

FIG. 7 shows that Wnt/β-catenin antagonism induce robust and specific MSC chondrogenesis. Wnt/β-catenin antagonism induces rapid chondrogenic differentiation by restricting osteogenic lineage commitment and enhancing pellet condensation through increasing N-cadherin expression and N-cadherin/β-catenin interactions at the AJ. The removal of TGFβ further avoids off-target specification towards osteogenesis/hypertrophy, fibrosis, and smooth muscle differentiation.

FIG. 8 shows that β-catenin expression levels after Wnt/β-catenin or TGFβ modulation in iPSC-MSCs undergoing chondrogenesis. Confocal immunofluorescence microscopy and fluorescence quantification of β-catenin in micromass-cultured iPSC-MSCs with the indicated modulators in chondrogenic induction medium for 1 day. Nuclei are labeled by DAPI. Scale bar, 5 μm. ChBM, chondrogenic basal medium without TGFβ. XAV, Wnt/β-catenin antagonist XAV939. CHIR, Wnt/β-catenin agonist CHIR99021. Data are represented as mean±S.D. *, p<0.05, **, p<0.01, ***, p<0.001.

### DETAILED DESCRIPTION OF THE EMBODIMENTS

The present application provides a composition for inducing chondrogenesis comprising an Wnt antagonist and a pharmaceutically acceptable carrier. The present application also provides a method for inducing chondrogenesis and a method for treating a cartilage-related disease by applying the above composition.

β-catenin/Wnt signaling, a major developmental and oncogenic pathway, is also important in skeletal related-tissue/organ development, especially for bone and cartilage tissue. We hypothesized that inhibition of the Wnt/β-catenin pathway may more precisely induce MSC chondrogenesis than TGFβ. To ascertain this, we utilized multiple sources of human MSCs—including iPSC-derived MSCs (iPSC-MSCs), ESC-derived MSCs (ESC-MSCs), and BM-MSCs—and also explored the role of AJ-β-catenin interactions in MSC chondrogenesis.

The antagonist of the Wnt signaling includes, for example, but not limited to Dickkopf (Dkk) proteins, secreted Frizzled-Related Proteins (sFRPs), and Wnt Inhibitory Factor-1 (WIF-1). In one embodiment, the Wnt antagonist can be XAV, DIF-3, iCRT3, ICG-001, IWP-2, IWP-4, Dkk, Soggy, sFRP, WIF-1, APCDD1, APCDD1L, Draxin, LMBR1L, Notum, SOST/Sclerostin, USAG1, or any combination thereof.

In the present application, the Wnt antagonist is preferably a Wnt/β-catenin antagonist. Some examples of the Wnt/β-catenin antagonist include, for example, but not limited to XAV, DIF-3 and iCRT3 In one embodiment, the Wnt/β-catenin antagonist is XAV.

In the present application, a stem cell can be further provided with the compositions or the methods. The stem cell can include totipotent stem cells, pluripotent stem cells, multipotent stem cells, and/or unipotent stem cells. The stem cell has an ability to differentiate into chondrocyte, for example, embryonic stem cell, induced pluripotent stem cell (iPSC), mesenchymal stem cell (MSC) and/or marrow stromal cell. In one preferred embodiment, the stem cell is mesenchymal stem cell (MSC). The MSC may be, for example, but not limited to iPSC-derived MSCs (iPSC-MSCs), ESC-derived MSCs (ESC-MSCs), BM-MSCs, adipose tissue-derived MSC, umbilical cord-derived MSC, placenta-derived MSC, uterus-derived MSC and dental pulp-derived MSC.

In the present application, the composition may further comprises a chondrocyte differentiation agent. The chondrocyte differentiation agent may be selected from the known agents. The chondrocyte differentiation agent may comprises, for example, but not limited to insulin growth factor (IGF1), Ruxolitinib, Tofacitinib, Baricitinib, angiopoietin-like 3 protein (ANGPTL3), oral salmon calcitonin, iNOS inhibitors, cholecalciferol, collagen hydrolyzate, bone morphogenetic protein 7 (BMP7), hyaluronic acid, a steroid, a non-steroidal anti-inflammatory agent (NSAID), TPX-100 and the like.

The present application also provides a method for inducing chondrogenesis comprising administering the composition to a subject, wherein the composition comprises an Wnt antagonist and a pharmaceutically acceptable carrier.

In one embodiment, the administration is in vitro or in vivo.

In one embodiment, the administration is local or systemic.

In some embodiments, the method can further comprise administering a stem cell having an ability to differentiate into chondrocyte to the subject. In one embodiment, the MSC can be co-cultured with the Wnt antagonist, and the mixture of the MSC and the Wnt antagonist is then administered to the subject. The subject can be a vertebrate. For example, the subject can be human, monkey, mice, rat, rabbit, pig, dog, cat and the like.

In some embodiments, the subject is the stem cell. The stem cell can be treated with the Wnt antagonist and the carrier to be induced to differentiate into chondrocyte.

The method may further comprises administering a chondrocyte differentiation agent to the subject. The chondrocyte differentiation agent can be administered prior to, simultaneously with, or after the administration of Wnt antagonist.

The present application further provides a method for treating a cartilage-related disease comprising administering a therapeutically effective amount of an Wnt antagonist and a pharmaceutically acceptable carrier to a subject.

In some embodiments, the method can further comprise administering a stem cell having an ability to differentiate into chondrocyte to the subject. The subject can be a vertebrate. For example, the subject can be human, monkey, mice, rat, rabbit, pig, dog, cat and the like.

The cartilage-related disease comprises, for example, but not limited to osteoarthritis, degenerative joint disease, osteochondritis dissecans, rheumatoid arthritis, articular joint injury, achondroplasia, and cartilage defects.

In one embodiment, the administration is in vitro or in vivo.

In one embodiment, the administration is local or systemic.

In one embodiment, the administration can be via injection, implantation, oral administration, and/or subcutaneous administration.

In the treating method of the present application, the therapeutically effective amount means the amount of the Wnt antagonist that, when administered to the subject, is sufficient to induce the chondrogenesis of the cells and/or the tissues, the formation of cartilage, and/or the treatment of cartilage-related disease. The therapeutically effective amount depends on the severity and course of the disease, previous treatment, the subject's health, weight, and response to medication, the judgment of the treating physician and the like. For example, the therapeutically effective amount of the Wnt antagonist can be 0.1 ug-100 mg per gram of the subject.

The treating method may further comprises administering a chondrocyte differentiation agent to the subject. The chondrocyte differentiation agent can be administered prior to, simultaneously with, or after the administration of Wnt antagonist.

In the present application, the pharmaceutically acceptable carrier can be one or more compatible conventional solid or liquid delivery systems as are well known in the art. Some examples of the pharmaceutically acceptable carrier are water, saline, phosphate buffer, sugars, starches, cellulose and its derivatives, powdered tragacanth, malt, gelatin, collagen, talc, stearic acids, magnesium stearate, calcium sulfate, vegetable oils, polyols, agar, alginic acids, and other suitable non-toxic substances and medicinal agents used in pharmaceutical formulations.

### EXAMPLES

Materials and Methods

Cell Culture

Human iPSC-MSCs were derived from iPSCs generated from fetal endothelial cells through lentiviral transduction of OCT-4 and SOX-2 (Ho et al., 2010), and human ESC-MSCs were derived from H1 (Wisconsin Alumni Research Foundation, Madison, Wis., USA) as previously reported (Peng et al., 2016; Wang et al., 2018; Yen et al., 2009). BM-MSCs were obtained from commercial sources (Promocell, Heidelberg, Germany). All MSCs were cultured and expanded in low-glucose Dulbecco's Modified Eagle's medium (DMEM) (Gibco-Thermo Fisher Scientific, MA, USA), with 10% FBS (Hyclone-Thermo Fisher Scientific) and 100 U/ml penicillin, 100 g/ml streptomycin, and 2 mM L-glutamine (all from Gibco-Thermo Fisher Scientific) as previously described (Pittenger et al., 1999; Yen et al., 2005).

In Vitro Chondrogenic Differentiation

In vitro chondrogenic differentiation using 3D pellet culture was performed as described previously (Liu et al., 2011; Wang et al., 2018; Yen et al., 2013), 2×10⁵ trypsinized were centrifuged 450×g for 10 minutes to form a pellet in expansion medium. After 16 hours, the medium was changed to chondrogenic basal medium (ChBM) consisting of low-glucose DMEM supplemented with 100 units/ml penicillin, 100 μg/ml streptomycin (all from Gibco-Thermo Fisher Scientific), 1% Insulin-transferrin-sodium selenite media supplement (Sigma-Aldrich, St Louis, Mo., USA), 2 mM L-glutamine (Gibco-Thermo Fisher Scientific), 10 μM L-Ascorbic acid 2-phosphate (Sigma-Aldrich), 100 nm dexamethasone (Sigma-Aldrich). Addition of TGFβ1 (10 ng/mL; R&D Systems, Minneapolis, Minn., USA), TGFβ3 (10 ng/mL; R&D Systems), XAV939 (XAV; 10 μM; Sigma-Aldrich) or CHIR99021 (CHIR; 10 μM; Sigma-Aldrich) to ChBM was performed as indicated. Cell pellets were harvested at the indicated day for analysis. Micromass culture-based chondrogenic differentiation was performed as previously reported, using 2×10⁷ cells/ml of MSCs suspended in expansion medium and 20-μl drops spotted in the center of each well of a 24-well culture plate (Craft et al., 2015; Greco et al., 2011). After adhesion for 1.5 hour in a humidified incubator at 37° C. with 5% CO₂, the medium was changed to ChBM with indicated regulators for chondrogenic induction.

Alcian Blue Staining and Quantification

Samples were washed with PBS and fixed with 4% paraformaldehyde, followed by pH 1.0 1% Alcian blue 8GX (Sigma-Aldrich) staining at room temperature overnight to detect sulfated proteoglycan matrix (Lev and Spicer, 1964). Quantification of Alcian blue staining extracted with 6M Guanidine-HCl was measured in 650 nm absorbance (Song et al., 2018).

Quantitative Real-Time PCR

Total RNA was isolated using TRI® reagent (Sigma-Aldrich), and quantified using NanoDrop spectrophotometer (Nyxor Biotech, Paris, France) (Wang et al., 2013). Reverse transcription was performed using RevertAid H Minus reverse transcriptase (Thermo Fisher Scientific), and PCR was performed on ABI PRISM 7500 system (Applied Biosystem, Foster City, Calif., USA) with SYBR Fast qPCR kit (KAPA Biosystems, Boston, Mass., USA). mRNA expression level was calculated by using the ΔΔCt method, and glyceraldehyde 3-phosphate dehydrogenase (GAPDH) level were used as housekeeping control. The results are normalized to ChBM to represent effects of indicated modulators. The sequences of primers for each gene are listed in Table 1.

TABLE 1
Primer sets for quantitative real-time PCR

| Gene Name | Forward | Reverse |
|---|---|---|
| SOX9 | 5′-TTTCCAAGACACAAACATGA-3′ | 5′-AAAGTCCAGTTTCTCGTTGA-3′ |
| COL2A1 | 5′-GGCAATAGCAGGTTCACGTACA-3′ | 5′-CGATAACAGTCTTGCCCCACTT-3′ |
| ACAN | 5′-TCGAGGACAGCGAGGCC-3′ | 5′-TCGAGGGTGTAGCGTGTAGAGA-3′ |
| RUNX2 | 5′-GGTTAATCTCCGCAGGTCACT-3′ | 5′-CACTGTGCTGAAGAGGCTGTT-3′ |
| AXIN2 | 5′-AGTGTGAGGTCCACGGAAAC-3′ | 5′-CTGGTGCAAAGACATAGCCA-3′ |
| GAPDH | 5′-CCACCCATGGCAAATTCCATGGCA-3′ | 5′-TCTAGACGGCAGGTCAGGTCCACC-3′ |

Immunofluorescent Staining

Immunofluorescent staining was performed as previously reported (Wang et al., 2013). Paraformaldehyde-fixed cell samples were permeabilized with 0.1% Triton X-100 and nonspecific binding was blocked by 5% bovine serum albumin Primary antibodies against alpha smooth muscle actin (αSMA) (Sigma-Aldrich), β-catenin (BD Biosciences, San Jose, Calif., USA) or N-cadherin (Abcam, Cambridge, UK) were stained with 1:100 dilution for 24 hours, followed by species specific secondary antibody incubation overnight. Fluorescent signals were acquired by confocal microscopy (Leica TCS SP5 II, Wetzlar, Germany) and analyzed by Image J (NIH, Maryland, USA).

Proximity Ligation Assay (PLA)

Micromass cultured iPSC-MSCs were used for PLA and performed according to the manufacturer's protocol (Sigma-Aldrich). Antibodies against N-cadherin (Abcam) and β-catenin (BD Biosciences) were used for recognition. After ligation and amplification, the PLA signals from each pair of probes in close proximity (<40 nm) were visualized by confocal microscope (Leica TCS SP5 II) and signal counts were analyzed by Image J.

In Vivo Chondrogenesis

All animal work was performed according to protocols approved by the Institutional Animal Care and Use Committee. In vivo ectopic chondrogenesis was performed as previously reported with modifications (Zhao et al., 2014). Mouse BM-MSCs were cultured as 3D pellets in ChBM alone or with addition of TGFβ3, CHIR, or XAV for 3 days, then subcutaneously transplanted to dorsal skin of wild-type C57BL/6J mice, with local injections of modulators every 3 days till the pellet sample was harvested at Day 20. Collected samples were then frozen in optimal cutting temperature (OCT) compound and sliced to 5 mm in thickness for histological staining.

Gene Expression and Pathway Analysis

Transcriptome data of human primary smooth muscle cells (GSE109859), MSCs (GSE128949 and GSE108186), chondrocytes (GSE68038), and osteoblasts (GSE121892) were obtained from public database. Datasets are merged to perform principal component analysis (PCA) using Partek® Genomics Suite (St. Louis, Mo., USA). Ranked Gene Set Enrichment Analysis (GSEA) was performed with software version 4.1.0 (Subramanian et al., 2005). Upstream Analysis were performed using IPA software (Qiagen, https://www.qiagenbioinformatics.com).

Quantification and Statistical Analysis

All data represent three replicates or more from separate experiments. Analysis of variance (ANOVA) followed by Tukey's post-hoc test was performed to evaluate significance for comparisons for multiple groups, with p<0.05 as significant. Analysis were performed using GraphPad Prism software and data are shown as mean±standard deviation (S.D.).

Results

Wnt/β-Catenin Antagonism Significantly Enhanced MSC Chondrogenesis while Agonism Resulted in the Opposite and Upregulation of the Master Osteogenic Transcription Factor Runx2

To investigate the effects of Wnt modulation on MSC chondrogenesis, we treated 3D pellet-cultured human MSCs—including iPSC-MSCs, ESC-MSCs and BM-MSCs—in complete chondrogenic induction medium (ChM) along with a small molecule Wnt/β-catenin agonist CHIR which inhibit GSK3β to disrupt the β-catenin destruction complex, or antagonist XAV, a tankyrase inhibitor which stabilizes the β-catenin destruction complex. TGFβ3 was utilized in ChM since it has been found to possess higher chondrogenic potential than TGFβ1 (Puetzer et al., 2010). We found that in ChM conditions, Wnt inhibition by XAV increased condensation of the pellets whereas agonism by CHIR decreased pellet integrity in all 3 sources of MSCs at Day 20.

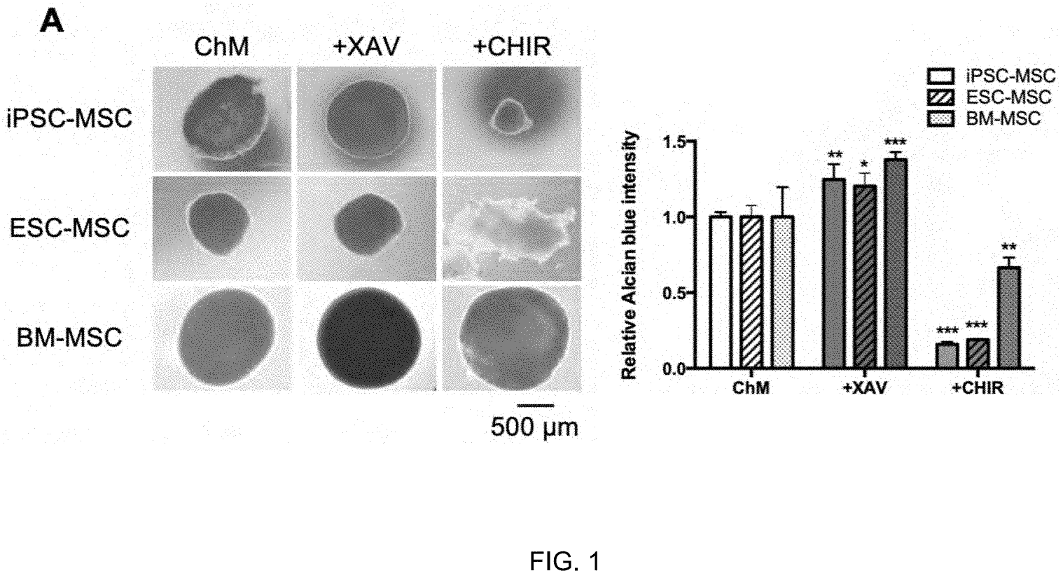

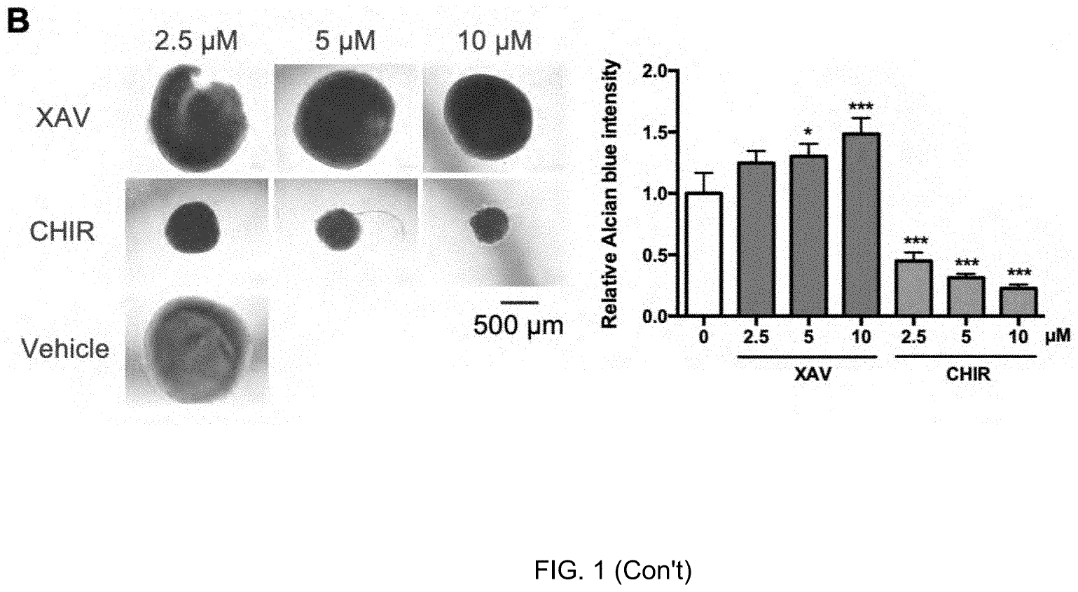

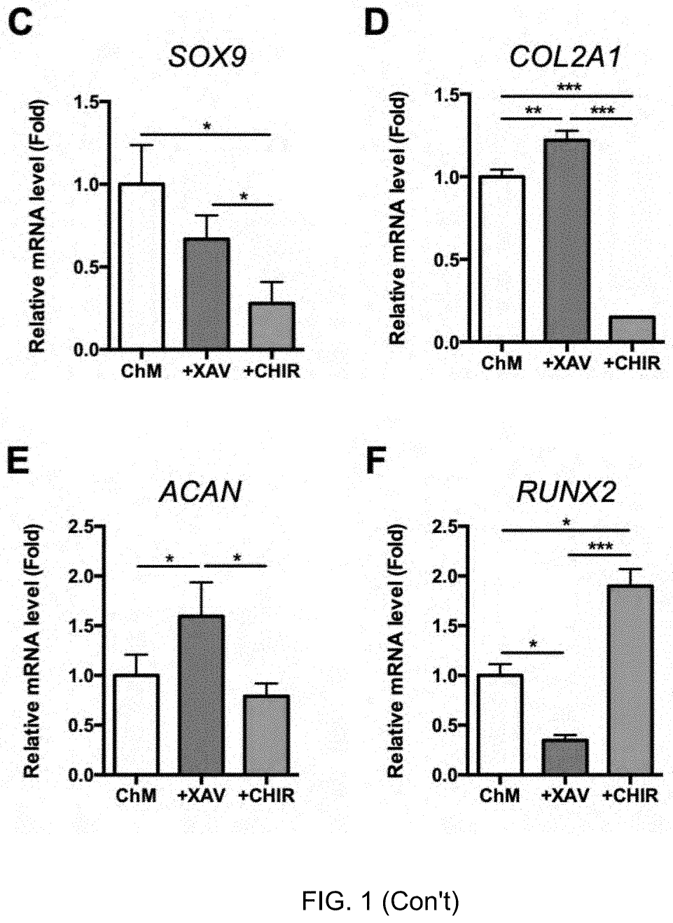

Quantification of glycosaminoglycans (GAGs) showed a significant increase in the expression of this structural extracellular matrix of cartilage with Wnt/β-catenin antagonism, whereas the opposite was seen with Wnt/β-catenin agonism (FIG. 1A). Since iPSC-MSCs can be patient-specific as well as continually derived, we focused on using this source in further studies. By observing morphological characteristics, including pellet size and integrity, and quantification of GAGs (FIG. 1B), we found a dose-dependent effect of Wnt/β-catenin modulation on MSC chondrogenesis. Analysis of chondrogenic gene expression levels after Wnt/β-catenin antagonism at Day 3 showed significant upregulation of two key chondrogenic genes collagen 2A1 (COL2A1) and aggrecan (ACAN) but not SOX9; Wnt/β-catenin agonism, on the other hand, resulted in significant downregulation of all 3 genes (FIG. 1C-1E). Interestingly, Wnt/β-catenin antagonism decreased expression of the master osteogenic transcription factor RUNX2 while agonism showed the opposite effect (FIG. 1F). These findings collectively demonstrate that Wnt antagonism significantly enhances MSC chondrogenic differentiation while agonism results in the opposite effect as well as upregulate the master osteogenic transcription factor RUNX2.

TGFβ Rapidly Increased Alpha Smooth Muscle Actin (αSMA) and RUNX2 Expression in MSCs During Chondrogenic Induction.

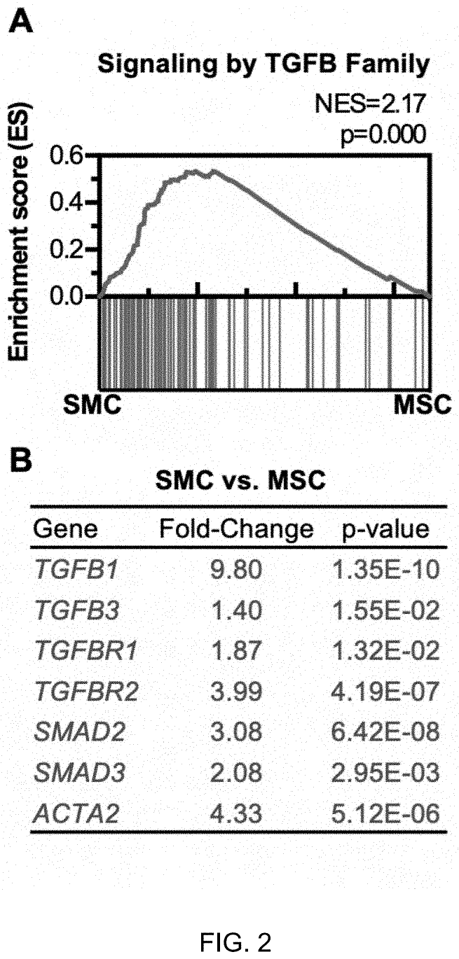

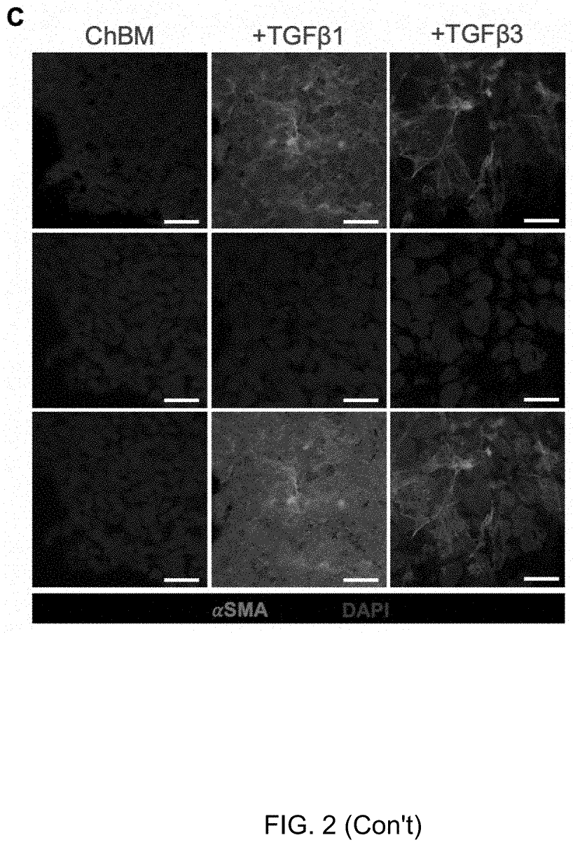

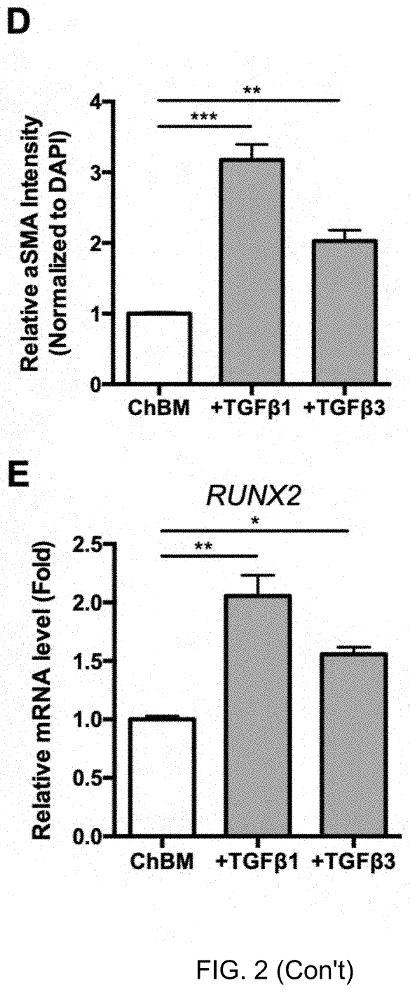

While TGFβ1 and TGFβ3 have long been used for MSC chondrogenesis, it is also well established that TGFβ can induce smooth muscle differentiation (Kurpinski et al., 2010) and upregulate an osteogenic program (Wu et al., 2016). By analyzing the transcriptomic data of human primary BM-MSCs and smooth muscle cells from public database (GSE128949 for human primary BM-MSCs; GSE109859 for human primary smooth muscle cells) with Ranked Gene Set Enrichment Analysis (GSEA), gene set of Signaling by TGFB Family Members was positively enriched in human BM-MSCs compared to smooth muscle cells with normalized enrichment score (NES) equals to 2.17 and the nominal p value less than 0.001 (FIG. 2A). Relative changes in mean expression levels also showed that the core components in TGFβ signaling (TGFB1, TGFB3 TGFBR1, TGFBR2, SAD2 and SMAD3) as well as αSMA (ACTA2), a marker of smooth muscle differentiation and also fibrosis (Li et al., 2011), were all significantly upregulated in human primary smooth muscle cells (SMC) compared to MSC (FIG. 2B). These results indicated that TGFβ signaling is positively correlated to the smooth muscle lineage. In addition to smooth muscle lineage, TGFβ/BMP signaling is also known to induce MSC osteogenic differentiation (Grafe et al., 2018). To further address the potential off-target effects of TGFβ in MSC chondrogenic differentiation, we assessed whether TGFβ upregulates expression of αSMA and/or RUNX2, the master osteogenic transcription factor, during MSC chondrogenesis Immunofluorescent staining of αSMA on Day 3 of MSC chondrogenic induction showed strong expression in both TGFβ-treated groups compared to chondrogenic basal medium (ChBM) which does not contain TGFβ (FIG. 2C), with quantitative fluorescent intensity demonstrating significantly higher αSMA expression (up to 3-fold) in TGFβ-treated groups compared to ChBM (FIG. 2D). RUNX2 mRNA expression was also significantly upregulated in TGFβ1- and TGFβ3-treated groups on Day 3 compared to ChBM (FIG. 2E). These results indicate that TGFβ rapidly induce non-chondrogenic lineages including smooth muscle and ossification during MSC chondrogenesis.

Wnt/β-Catenin Antagonism Alone Induced More Rapid MSC Chondrogenesis than TGFβ

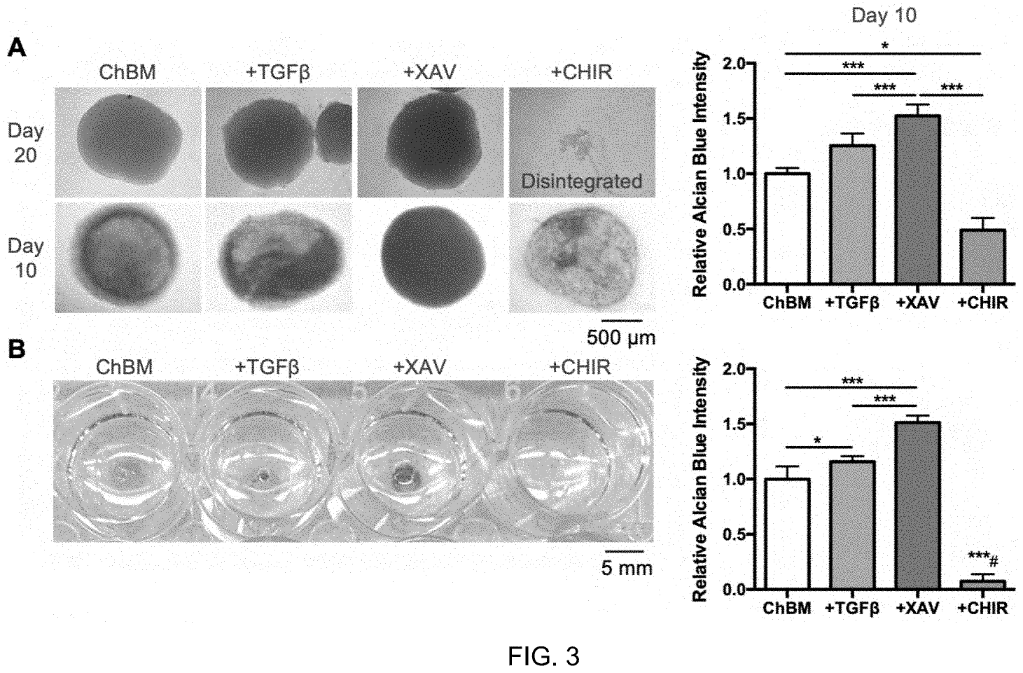

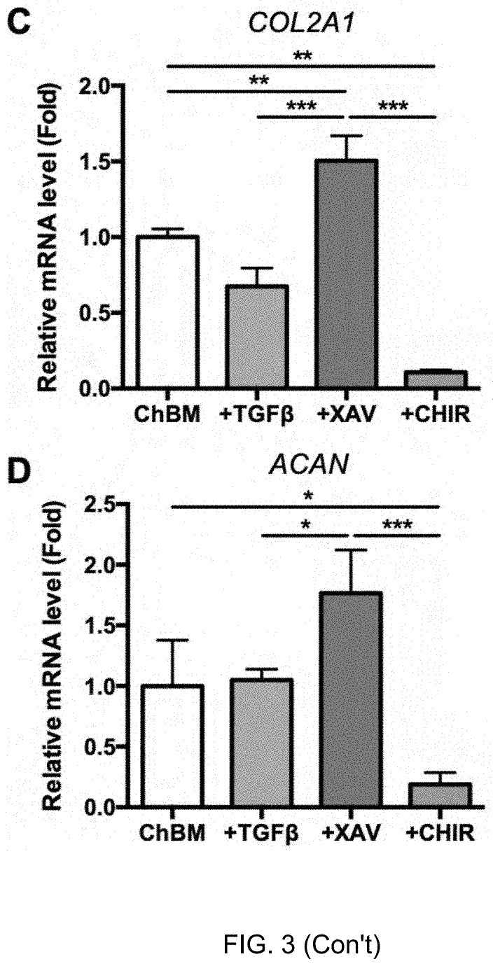

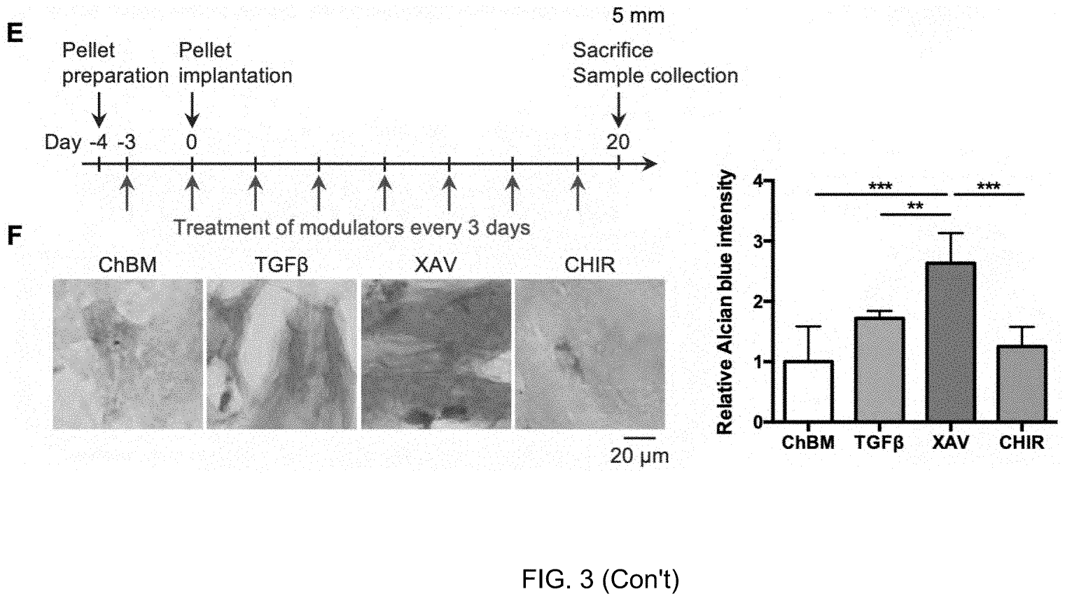

Since Wnt/β-catenin antagonism strongly enhances MSC chondrogenesis and TGFβ agonism significantly induce other non-chondrogenic lineage markers during this process, we examined the possibility of replacing TGFβ completely with Wnt/β-catenin antagonism to achieve more specific MSC chondrogenic differentiation. After 20 days of chondrogenic differentiation, MSC pellets treated with either TGFβ3 or XAV formed well-condensed spheres and strong Alcian Blue staining compared to ChBM, while pellets treated with CHIR did not undergo condensation but became disintegrated at the end of the culture time period (FIG. 3A, top panel). Strikingly, we found that GAG production as evidenced by Alcian Blue staining was more rapidly induced in pellets cultured with XAV than TGFβ3 at Day 10, approximately halfway through the full differentiation time period (FIG. 3A, bottom panel). This was verified with quantification of GAG production at Day 10 in which significant increases in MSC pellets cultured with XAV was seen, not only when compared to ChBM culture but also to TGFβ3 treatment, while CHIR treatment resulted in minimal GAG production (FIG. 3A, right panel). We further validated these findings using micromass culture, which is a more convenient method to induce chondrogenic differentiation in standard 2D culture condition and useful for in vitro high-throughput drug screening (Greco et al., 2011).

Similar to 3D pellet culture, micromass culture of MSCs with XAV treatment at Day 10 demonstrated better structural condensation and stronger Alcian Blue intensity compared to ChBM culture and even TGFβ3-treated culture, while CHIR treatment resulted in minimal evidence of any micromass structure or Alcian Blue staining. Quantitation of GAG production was in line with the morphologic findings, with XAV treatment resulting in the highest production of GAG (FIG. 3B). XAV treatment also most significantly upregulated expression of both chondrogenic genes COL2A1 and ACAN, while TGFβ3 treatment did not increase expression of either gene and CHIR treatment resulted in minimal expression of either genes at Day 3 (FIGS. 3C and D).

To validate our in vitro data, we performed subcutaneous transplantation into wildtype mice of mouse BM-MSCs cultured in ChBM medium with injections of various modulators (TGFβ3, XAV, or CHIR) every three days for 20 days (FIG. 3E). Histological analyses of in vivo differentiated samples demonstrated that the highest level of GAG production was in the XAV-treated group; moreover, Wnt/β-catenin antagonism significantly increased GAG production compared to TGFβ3 agonism (FIG. 3F). These results indicated that Wnt inhibition significantly enhances MSC chondrogenesis in vivo. All these results demonstrate that replacement of TGFβ with Wnt/β-catenin antagonism achieves more rapid and specific MSC chondrogenesis.

Wnt/β-Catenin Antagonism but not TGFβ Agonism During MSC Chondrogenesis Decreased Canonical Wnt/β-Catenin Transcriptional Activity Including RUNX2 Expression

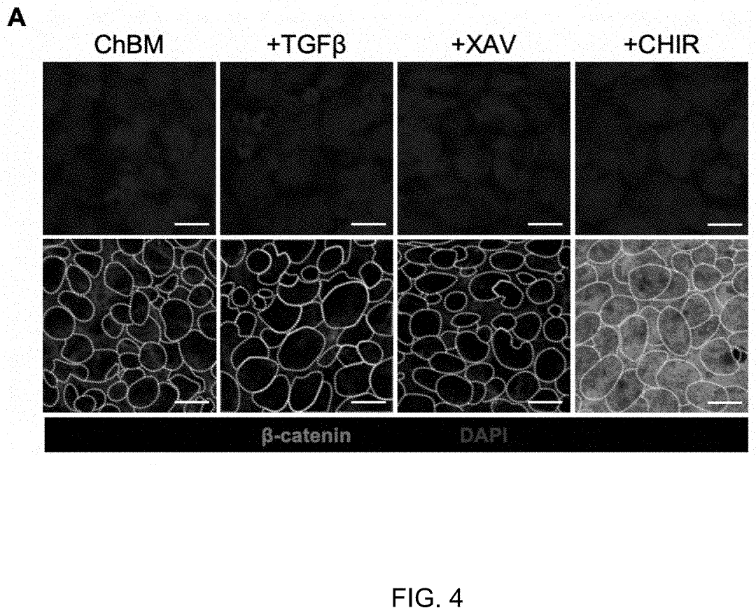

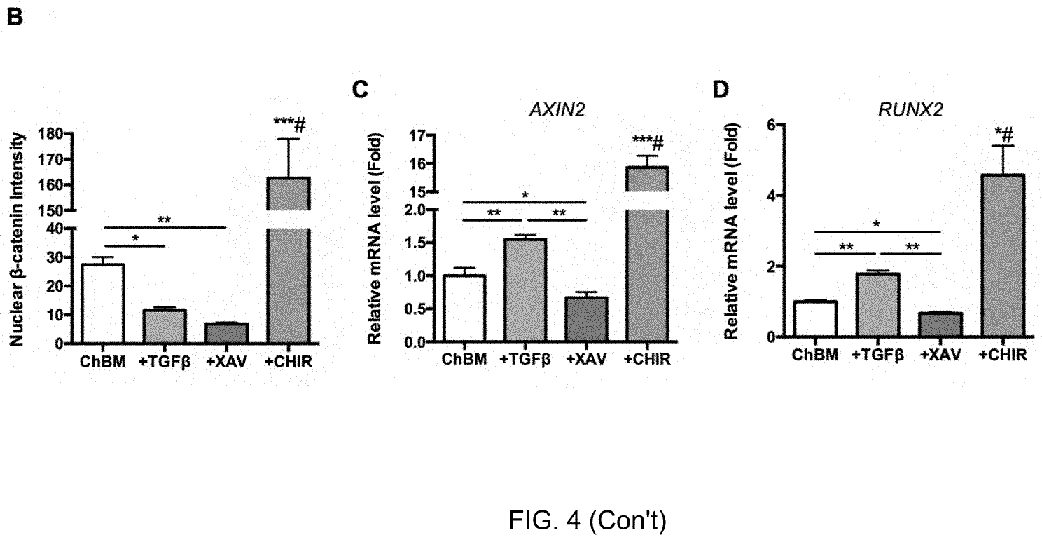

The significant enhancement of MSC chondrogenesis with Wnt/β-catenin antagonism alone led us to investigate the role of the canonical Wnt/β-catenin pathway during the differentiation process. As a transcription factor, β-catenin undergoes translocation from the cytoplasm to the nucleus when activated. Using immunofluorescent staining and quantification, we found that pellet-cultured MSCs treated with XAV had the lowest nuclear β-catenin levels compared to control ChBM culture or TGFβ3 treatment, while CHIR treatment dramatically increased nuclear β-catenin levels (FIGS. 4A and 4B), which was expected since CHIR is known to strongly induce β-catenin transcriptional activity (Narcisi et al., 2016). Assessment of β-catenin pathway downstream gene expression levels demonstrated that after XAV treatment, expression of AXIN2, a well-established β-catenin-activated downstream gene, and the osteogenic master transcription factor RUNX2 were both significantly decreased, whereas CHIR treatment strongly increased expression of both genes as expected (FIGS. 4C and 4D). Surprisingly, TGFβ3 treatment resulted in increased expression of not only RUNX2 but AXIN2 as well, implicating a weak agonistic effect of this factor on canonical Wnt/β-catenin pathway. These findings demonstrate that inhibition of Wnt/β-catenin transcriptional activity is critical for MSC chondrogenesis, and that TGFβ treatment may not be optimal during this process due to its weak agonism for the pathway.

Wnt/β-Catenin Antagonism but not TGFβ Agonism Increased N-Cadherin Expression and Interactions with β-Catenin at AJs as Well as Enhancing Actin Cytoskeleton-Mediated Condensation

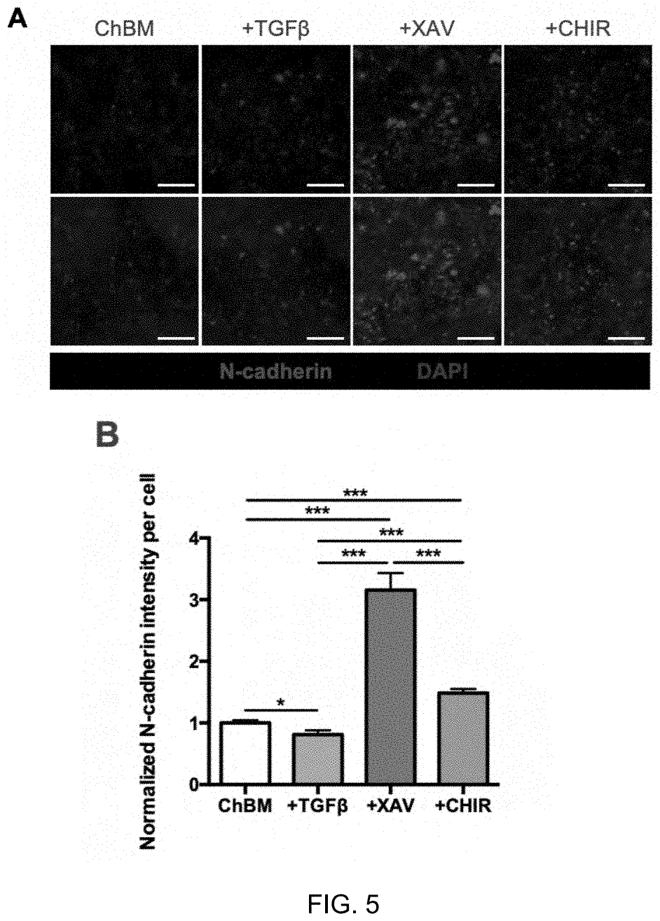

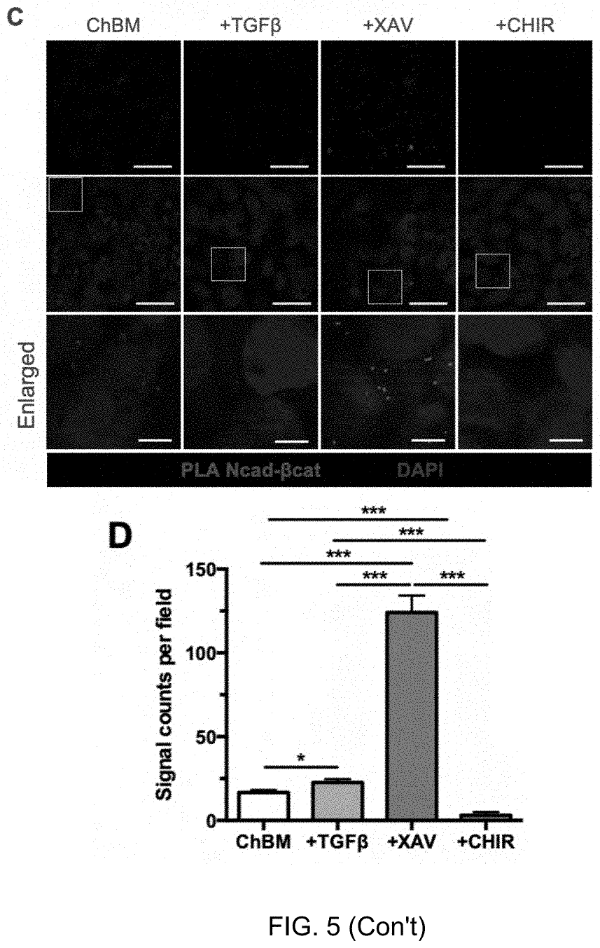

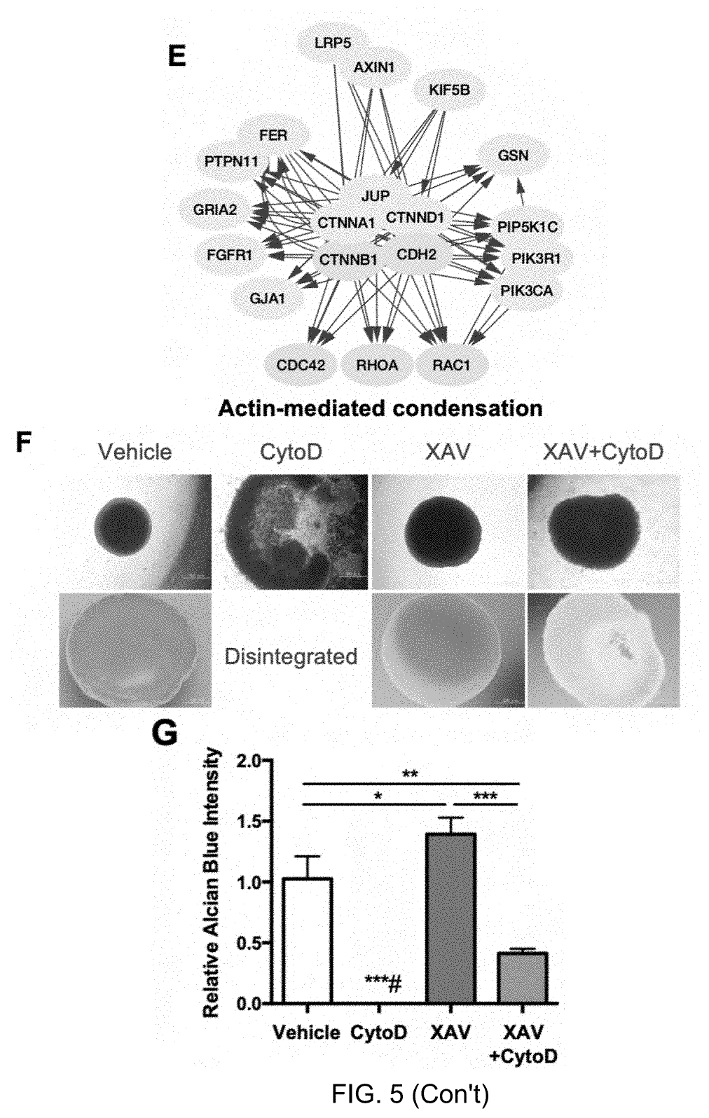

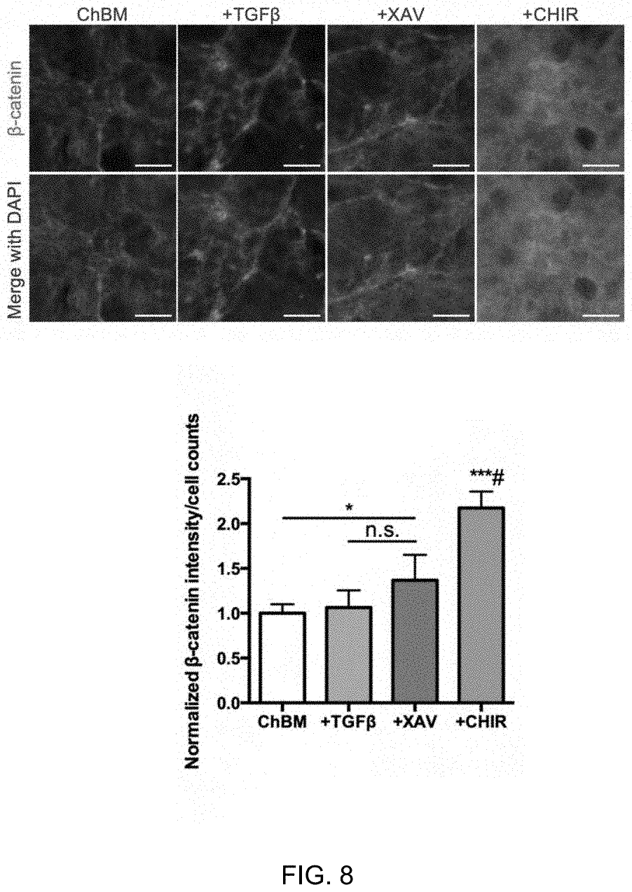

In addition to its role as a transcription factor, β-catenin is also a component of the AJ, a key structure of cell-cell adhesion which is a critical aspect during cartilage condensation (Alimperti and Andreadis, 2015). To investigate how Wnt/β-catenin modulation during MSC chondrogenesis affect AJs, we performed immunofluorescent staining for N-cadherin, a major AJ component in mesenchymal cell types like MSCs which is also essential during chondrogenesis (Quintana et al., 2009). We found N-cadherin expression to be strongly and most significantly upregulated in MSC micromass culture with XAV treatment after 24 hours, compared to all other conditions (FIGS. 5A and 5B). To further investigate whether there were increased interactions between β-catenin and the increased N-cadherin levels induced by Wnt/β-catenin antagonism, we performed proximity ligation assay (PLA) between these two molecules. We found that only XAV treatment during MSC chondrogenesis could significantly increase N-cadherin/β-catenin interactions as evidenced by increased PLA signals (FIGS. 5C and 5D). Interestingly, while CHIR treatment resulted in the strongest expression of β-catenin (FIG. 8), there was minimal PLA signal detected, indicating little interaction/colocalization between N-cadherin and β-catenin (FIGS. 5C and 5D). These findings demonstrate that Wnt inhibition enhances MSC chondrogenic differentiation through increasing N-cadherin levels and interaction/colocalization between N-cadherin and β-catenin at AJs.

To validate interactions between N-cadherin and β-catenin in MSC chondrogenic differentiation, we first examined the N-cadherin pathway using the Pathway Interaction Database (PID_NCADHERIN_PATHWAY) and found that along with N-cadherin (CDH2), β-catenin (CTNNB1) is a core participant within the signaling pathway, along with numerous components of the AJ: α-catenin (CTNNA1), p120/δ-catenin (CTNND1), and plakoglobin (JUP). Moreover, the three major GTPases responsible for actin cytoskeleton organization—RhoA, CDC42, and Racl—are all found as downstream effectors in the signaling pathway (FIG. 5E), demonstrating that actin cytoskeleton organization is highly regulated by the N-cadherin signaling pathway. To validate the bioinformatics analyses, during MSC chondrogenesis we treated with cytochalasin D (CytoD), an inhibitor of actin polymerization, which significantly disrupted the condensation process with the pellets completely disintegrated by Day 10. In contrast, cells treated with XAV+CytoD partially maintained the pellet structure and chondrogenic differentiation (FIGS. 5F and 5G). All these results suggest that Wnt/β-catenin antagonism promotes MSC chondrogenic differentiation by increasing N-cadherin expression and its interactions with β-catenin at AJs as well as enhancing actin cytoskeleton-mediated condensation.

Significant Downregulation of Wnt/β-Catenin and TGFβ-Related Pathways in Transcriptomes of Human Primary Chondrocytes Compared to Osteoblasts

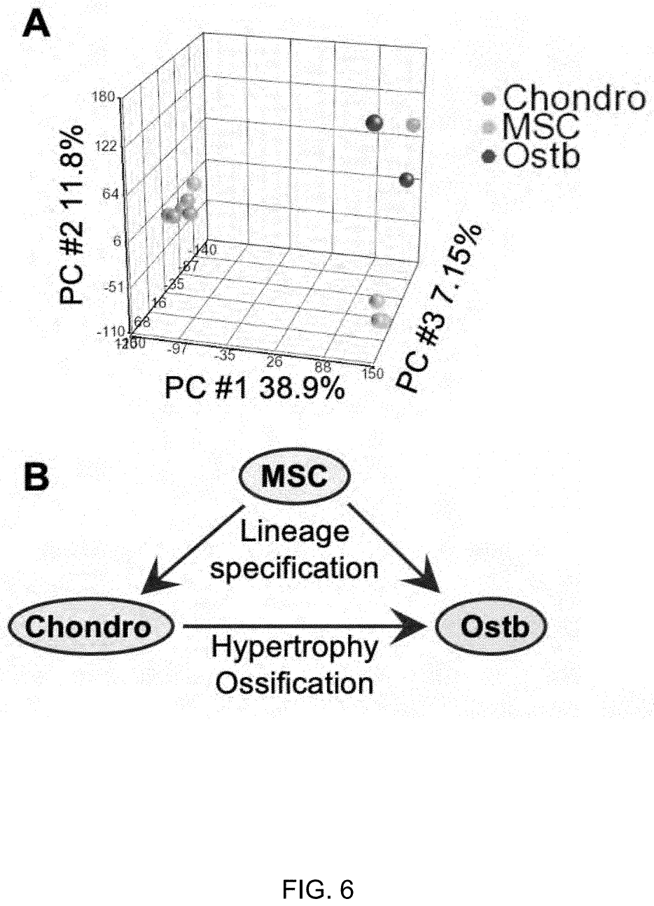

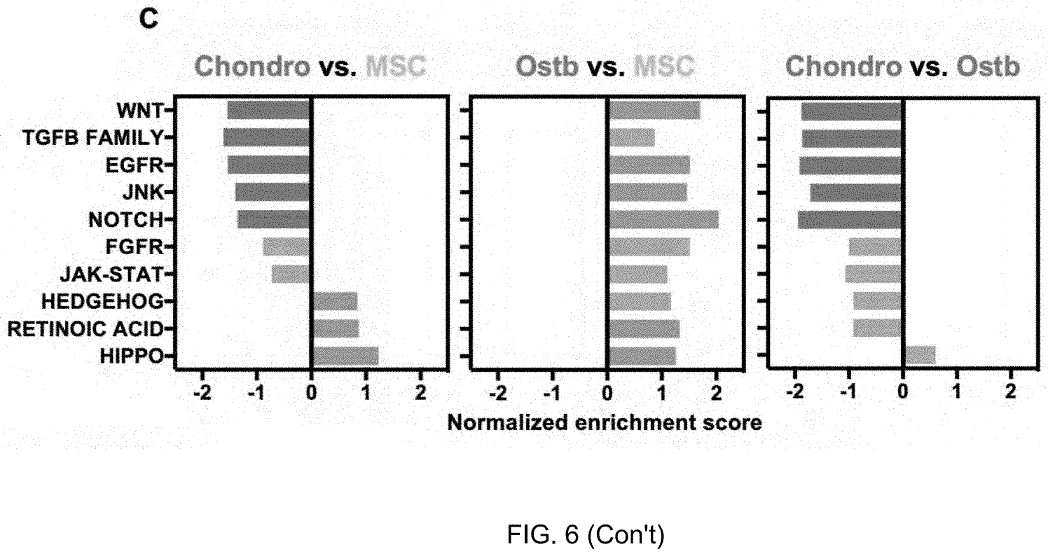

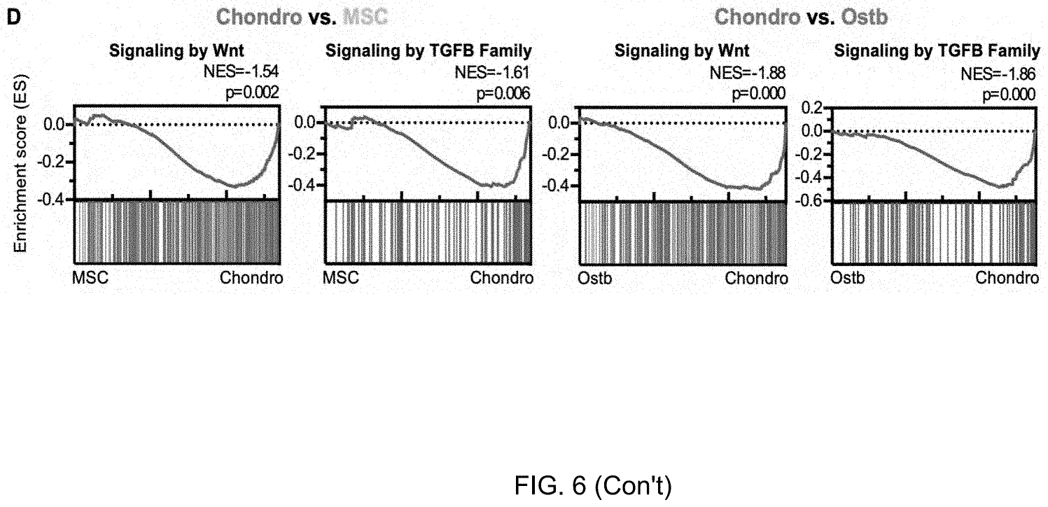

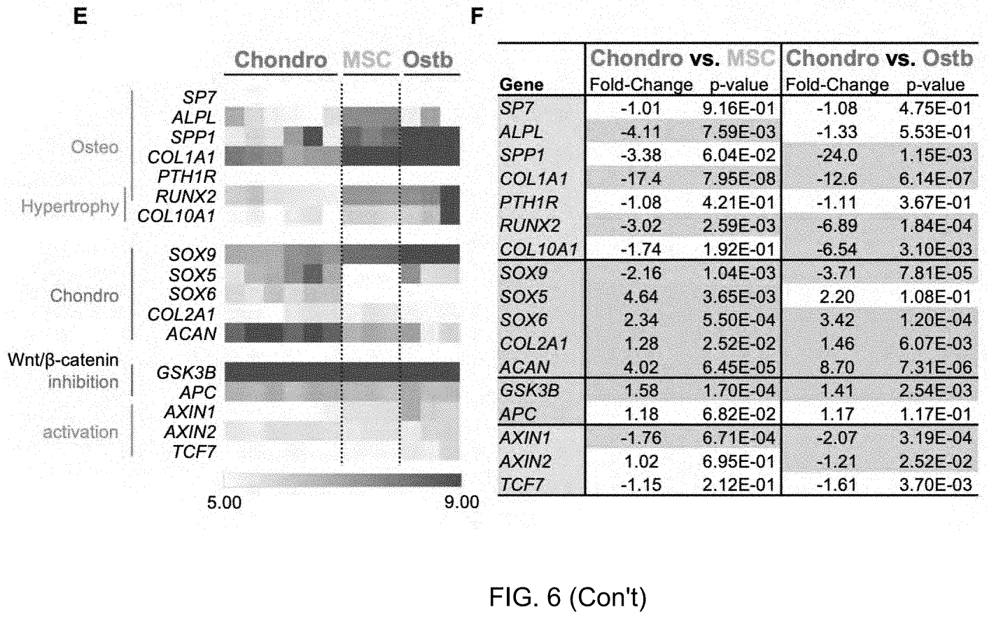

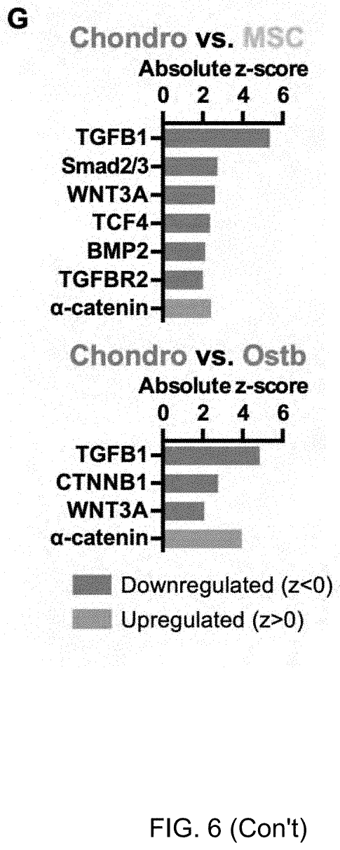

To assess the physiological relevance of our findings, we performed bioinformatics analyses using human primary BM-MSC, chondrocyte, and osteoblast transcriptome data from public database (GSE108186 for human primary BM-MSCs; GSE68038 for human primary chondrocytes; and GSE121892 for human primary osteoblasts). Initial analyses using principal component analysis (PCA) demonstrated that chondrocytes, osteoblasts, and MSCs are three highly distinct populations (FIG. 6A). We then evaluated the transcriptomic changes of MSC lineage specification toward chondrogenic and osteogenic lineages by comparing mRNA expression profiles of chondrocytes and osteoblasts to MSCs (Chondro vs. MSC and Ostb vs. MSC, respectively). To uncover genes and pathways relevant to cartilage maintenance but not hypertrophy and ossification, we compared the transcriptomic profiles of chondrocytes to osteoblasts (Chondro vs. Ostb) (FIG. 6B). GSEA based on major developmental pathways (Perrimon et al., 2012) showed that both Wnt and TGFβ family signaling are negatively enriched in Chondro vs. MSC (FIG. 6C, left panel) and Chondro vs. Ostb (FIG. 6C, right panel); in contrast, in Ostb vs. MSC, Wnt signaling is positively enriched and TGFβ family signaling is non-significantly enriched (FIG. 6C, middle panel). These findings implicated that the strategy of Wnt antagonism with removal of TGFβ for MSC chondrogenesis is physiologically relevant and less likely to result in hypertrophy/ossification. In addition, enrichment plots of Wnt and TGFβ family signaling pathways in Chondro vs. MSC showed that both pathways are significantly enriched (p<0.05) with normalized enrichment score (NES) of −1.54 and −1.61, respectively (FIG. 6D, left two panels). Similarly, in Chondro vs. Ostb, Wnt and TGFβ family signaling were also significantly enriched with NES of −1.54 and −1.61, respectively, (FIG. 6D, right two panels). We then analyzed by cell type the mRNA expression levels of specific genes in the canonical Wnt/β-catenin pathway, during chondrogenesis, as well as during osteogenesis for further validation of the GSEA results (FIG. 6E). To better assess the directionality of expression for each gene, we compared the changes of Robust Multi-array Average (RMA) levels of specific genes in human chondrocytes to that of MSCs (Chondro vs. MSC) and osteoblasts (Chondro vs. Ostb) (FIG. 6F). The results showed that the osteogenic/hypertrophy genes RUNX2, COL1A1 and ALPL were significantly downregulated in primary chondrocytes compared to MSCs, and RUNX2, ALPL, SPP1, COL1A1 and COL10A1 were downregulated in chondrocytes compared to osteoblasts. In contrast, chondrogenic genes including SOX5, SOX6, COL2A1 and ACAN were more highly expressed in chondrocytes than MSCs or osteoblasts, with the surprising exception of SOX9 which was more highly expressed in MSCs and osteoblasts than in chondrocytes. GSK3B, an inhibitory gene of the Wnt/β-catenin pathways, were more highly expressed in primary chondrocytes, whereas Wnt/β-catenin downstream genes AXIN1, AXIN2 were less expressed in chondrocytes compared to MSCs or osteoblasts.

Using Upstream Regulator Analysis in Ingenuity Pathway Analysis (IPA) which can provide a more causal relationship between transcription factors/master regulators and downstream pathways (Kramer et al., 2014), we found that TGFβ1, Smad2/3, WNT3A, TCF4, BMP2, and TGFBR2 were all predicted to be downregulated transcription factors/master regulators in human primary chondrocytes compared to MSCs. Furthermore, TGFβ1, CTNNB1, and WNT3A were predicted to be downregulated regulators in human primary chondrocytes compared to osteoblasts. Specifically, α-catenin—a core components of the AJ—was predicted as an upregulated regulator in chondrocytes compared to both MSCs and osteoblasts (FIG. 6G). All these bioinformatic results were in line with our data demonstrating participation of AJs in WNT/β-catenin antagonism-induced chondrogenesis (FIG. 5). Overall, these results underscore that the Wnt/β-catenin pathway as well as the TGFβ pathway is downregulated in primary human chondrocytes compared to osteoblasts and MSCs.

Discussion

MSC therapy likely offers the best possibility of a curative treatment for cartilage and joint diseases, which currently lacks such significant disease-modifying treatments. However, the relatively more difficult 3D pellet differentiation protocol and the need for using a protein-based growth factor, TGFβ, continue to be obstacles for robust clinical applications. TGFβ is a cytokine with complex functions, and has pleomorphic roles in specification of multiple mesenchymal lineages. In addition, this cytokine is part of a superfamily of ligands which includes the BMP subfamily, factors essential in ossification and bone biology. Therefore, it may be difficult to avoid osteogenic specification with use of proteins from this family during chondrogenic differentiation. While some early in vitro stem cell differentiation studies did not consistently find Wnt inhibition to promote chondrogenesis, these reports utilized biologically derived Wnt inhibitory ligands which are known to have off-target effects as well as potency issues (Im and Quan, 2010; Yano et al., 2005). In addition, some studies utilized non-MSCs which may not be the appropriate system to investigate chondrogenic lineage specification through Wnt/β-catenin (Yano et al., 2005). Using highly potent small molecule agonists and antagonists of the Wnt/β-catenin pathway, we demonstrated that Wnt/β-catenin antagonism efficiently induce in vitro chondrogenesis in human MSCs from multiple sources, and in vivo using murine BM-MSCs. Moreover, in comparison with TGFβ3, the most potent member of the family for induction of chondrogenesis, Wnt/β-catenin antagonism more rapidly induced chondrogenesis without inducing other non-chondrogenic, off-target, lineages. Our findings are further supported by transcriptome analysis of primary human MSCs, chondrocytes, and osteoblasts, which demonstrated that the Wnt/β-catenin and TGFβ family signaling pathways were downregulated in chondrocytes relative to the other 2 cell types (FIG. 6C-6G). Our findings therefore strongly implicated that Wnt/β-catenin antagonism using potent small molecules can efficiently and more precisely promotes MSC chondrogenesis than TGFβ.

We found that the efficient chondrogenic commitment mediated by Wnt/β-catenin antagonism involved strong upregulation of N-cadherin expression and N-cadherin/β-catenin interaction at the AJ which enhanced pellet condensation (FIG. 5), a critical step in chondrogenesis and cartilage formation (Sart et al., 2014). Since initiation of chondrogenic induction of MSCs requires better cell-cell interaction rather than cell-matrix adhesion, non-adhesive 3D pellet culture or micromass culture are considered as standard methods to induce MSC chondrogenic differentiation. Previous reports showed that neutralization of N-cadherin results in the inability of mesenchymal cells to condense and therefore inhibits subsequent chondrogenesis (Oberlender and Tuan, 1994); conversely, addition of N-cadherin biomimetic peptides can enhance neocartilage formation by human MSCs (Bian et al., 2013). These findings demonstrated the crucial role of N-cadherin-mediated cell condensation in promoting chondrogenic differentiation. We found that expression of N-cadherin, and interactions between N-cadherin and β-catenin were significantly upregulated with Wnt inhibition during MSC chondrogenic differentiation (FIG. 5), implying that Wnt/β-catenin antagonism can increase N-cadherin levels and cell condensation, both critical processes for chondrogenesis. Moreover, disruption of actin polymerization—also a critical process during in vivo condensation (Ray and Chapman, 2015)—by CytoD was partially rescued with Wnt/β-catenin antagonism (FIGS. 5F and 5G), demonstrating the key role of this pathway on multiple aspects of chondrogenesis. Even more striking, using Upstream Regulator Analysis which predict involvement of transcription factors/master regulators, α-catenin was predicted as an upregulated factor, whereas TGFβ as well as Wnt/β-catenin members were predicted to be downregulated in primary chondrocytes compared to MSCs as well as osteoblasts (FIG. 6G). Our findings therefore strongly implicate the critical role of β-catenin as a structural protein as well as further support the importance of N-cadherin in the AJ and the cytoskeleton-mediated condensation during MSC chondrogenesis.

Our study revealed the many off-target lineages specification by TGFβ during MSC chondrogenesis. One of the most important molecules/pathways in developmental biology, TGFβ is known to be involved in specification of multiple mesodermal lineages, as well as mediate pathological fibrotic processes. In the development of the skeletal system, TGFβ not only induces chondrogenic differentiation and modulates the process of hypertrophy (Mueller et al., 2010) but also osteogenic differentiation as well (Wu et al., 2016). In addition, TGFβ promotes differentiation of MSCs into smooth muscle cells (Kurpinski et al., 2010); this was clearly reflected in our transcriptomic analysis which showed that signaling by TGFβ family was highly enriched in SMCs compared to MSCs (FIGS. 2A and 2B). Interestingly, the evidence for involvement of TGFβ signaling in chondrogenesis appears to be largely in vitro, since no defect in cartilage formation seen in either TGFβ1- or TGFβ3-deficient mice (Wang et al., 2014). Despite the well documented evidence on the pleomorphic effects of TGFβ on multiple mesenchymal lineages, no study has concurrently evaluated commitment into these lineages. We found that even under chondrogenic 3D pellet culture conditions, TGFβ increased the expression of both the smooth muscle-lineage marker αSMA and the osteogenic/hypertrophic marker RUNX2 (FIG. 2). Such significant off-target lineage specifications may contribute to a lower chondrogenic differentiation efficiency, and be responsible for ossification and/or fibrosis observed in transplanted chondrocytes derived from TGFβ-differentiated MSCs (Lee and Wang, 2017; Pelttari et al., 2006). While one recent study reported that removal of TGFβ in the late stage of MSC chondrogenic differentiation reduced hypertrophic cartilage formation (Deng et al., 2019), we found that addition of TGFβ rapidly upregulated a canonical Wnt/β-catenin downstream gene AXIN2 in addition to RUNX2 as early as Day 3 of the differentiation period (FIGS. 4C and 4D), which is in line with previous reports that TGFβ is a weak agonist for Wnt/β-catenin signaling (Cleary et al., 2015). Our findings along with previous transgenic mice data collectively support that Wnt/β-catenin antagonism rather than TGFβ agonism may be the most critical and appropriate pathway to efficiently and specifically induce MSC chondrogenic lineage commitment.

The master transcription factor for chondrogenesis is SOX9 (Bi et al., 1999), however, we did not find further upregulation of this transcription factor with Wnt inhibition during MSC chondrogenic induction using 3D pellet culture (FIG. 1C), unlike the significant upregulation of more downstream chondrogenic genes COL2A1 and ACAN (FIGS. 1D and 1E). Our in vitro data was surprisingly corroborated in transcriptome analysis of human samples, in which SOX9 was downregulated in chondrocytes compared to osteoblasts and undifferentiated MSCs (FIGS. 6E and 6F). Similar to our data, a discrepant trend in expression levels between SOX9 and mature chondrogenic markers including COL2A1 and ACAN has been reported previously (Adkar et al., 2019; Lorda-Diez et al., 2009; Yang et al., 2012). These findings collectively strongly suggest that high levels of Sox9 expression are a necessary event in development and early stages of chondrogenesis, but less evident at later stages including in vitro differentiation in somatic progenitors/stem cells such as MSCs.

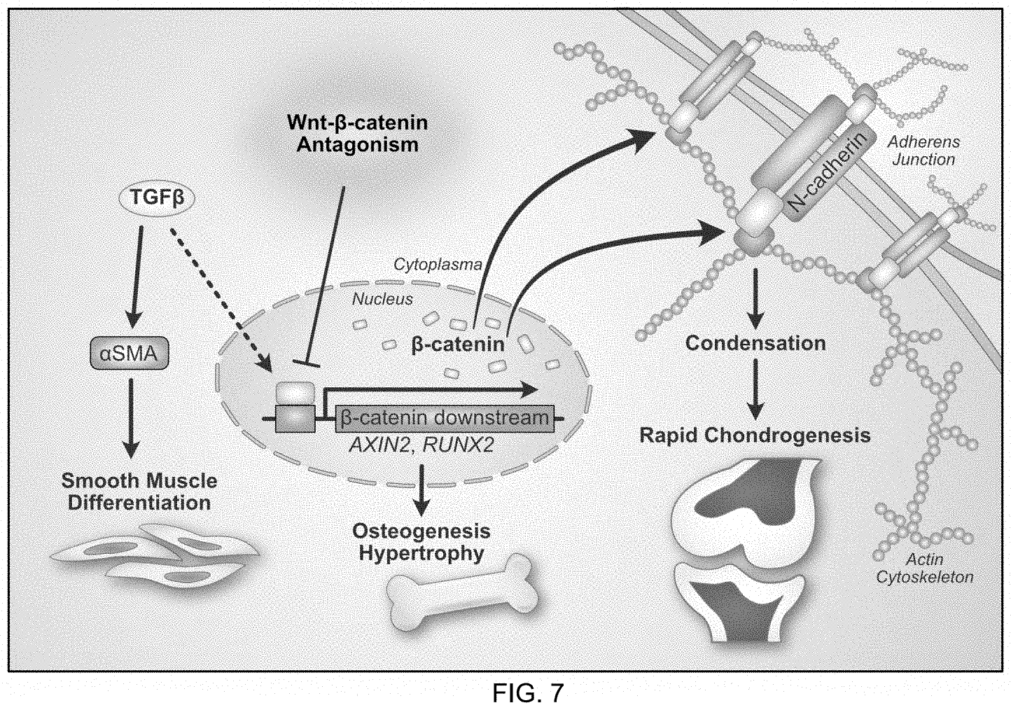

In summary, using multiple sources of human MSCs including ESC-MSCs and iPSC-MSCs we found that replacement of TGFβ agonism with Wnt/β-catenin antagonism resulted in robust and specific in vitro and in vivo MSC chondrogenesis by eliminating off-target lineage specification into osteogenesis/hypertrophic cartilage and smooth muscle. Wnt/β-catenin antagonism also more efficiently induced MSC chondrogenesis by increasing N-cadherin levels as well as N-cadherin-β-catenin interactions at the AJ to enhance condensation (FIG. 7). These findings are also corroborated by bioinformatic analysis of human primary MSC, chondrocyte, and osteoblast transcriptomes, in which downregulation of both Wnt/β-catenin and TGFβ pathways, along with upregulation of α-catenin-related processes was seen in primary chondrocytes compared to MSCs and osteoblasts. Our study underscores the importance of structural modification in MSC chondrogenesis, as well as having a thorough understanding of key developmental pathways in lineage specification. The capacity to use small molecules rather than a protein growth factor for stem cell differentiation is also more cost-effective, and therefore highly relevant for translational application.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A composition for inducing chondrogenesis comprising an Wnt antagonist and a pharmaceutically acceptable carrier.

2. The composition of claim 1, wherein the Wnt antagonist is Wnt/β-catenin antagonist.

3. The composition of claim 1, wherein the Wnt antagonist is selected from a group consisting of XAV, DIF-3, iCRT3, ICG-001, IWP-2, IWP-4, Dkk, Soggy, sFRP, WIF-1, APCDD1, APCDD1L, Draxin, IGFBP-4, LMBR1L, Notum, SOST/Sclerostin, and USAG1.

4. The composition of claim 1, wherein the Wnt/β-catenin antagonist comprises XAV, DIF-3 and iCRT3.

5. The composition of claim 1, which further comprises a stem cell having an ability to differentiate into chondrocyte.

6. The composition of claim 5, wherein the stem cell is mesenchymal stem cell (MSC).

7. A method for inducing chondrogenesis comprising administering an Wnt antagonist and a pharmaceutically acceptable carrier to a subject.

8. The method of claim 7, wherein the Wnt antagonist is selected from a group consisting of XAV, Dkk, Soggy, sFRP, WIF-1, APCDD1, APCDD1L, Draxin, IGFBP-4, LMBR1L, Notum, SOST/Sclerostin, and USAG1.

9. The method of claim 7, which further comprises administering a stem cell having an ability to differentiate into chondrocyte to the subject.

10. The method of claim 9, wherein the stem cell is mesenchymal stem cell (MSC).

11. The method of claim 10, wherein the MSC is co-cultured with the Wnt antagonist before the administration to the subject.

12. The method of claim 7, wherein the subject is a stem cell having an ability to differentiate into chondrocyte.

13. A method for treating a cartilage-related disease comprising administering a therapeutically effective amount of an Wnt antagonist and a pharmaceutically acceptable carrier to a subject.

14. The method of claim 13, wherein the Wnt antagonist is selected from a group consisting of XAV, Dkk, Soggy, sFRP, WIF-1, APCDD1, APCDD1L, Draxin, IGFBP-4, LMBR1L, Notum, SOST/Sclerostin, and USAG1.

15. The method of claim 13, which further comprises administering a stem cell having an ability to differentiate into chondrocyte to the subject.

16. The method of claim 15, wherein the stem cell is mesenchymal stem cell (MSC).

17. The method of claim 16, wherein the MSC is co-cultured with the Wnt antagonist before the administration to the subject.

18. The method of claim 13, wherein the cartilage-related disease comprises osteoarthritis, degenerative joint disease, osteochondritis dissecans, rheumatoid arthritis, articular joint injury, achondroplasia, and/or cartilage defects.

19. The method of claim 13, wherein the administration is local or systemic.

20. The method of claim 13, wherein the administration is in vivo or in vitro.
